(19) United States
(12) Patent Application Publication
(10) Pub. No.: US 2003/0078659 A1
(43) Pub. Date: Apr. 24, 2003

(54) Graft element

(76) Inventor: Jun Yang, Dove Canyon, CA (US)

Correspondence Address: Raymond Sun, 12420 Woodhall Way, Tustin, CA, 92782, US

(21) Appl. No.: 10/000,518

(22) Filed: Oct. 23, 2001

(51) Int. Cl. A61F 2/08

(52) U.S. Cl. 623/13.17; 623/902; 623/901

(57) ABSTRACT

A method of forming an elongated graft element includes procuring a tubular tissue from a mammal, with the tubular tissue having a longitudinal axis, and a distal edge and a proximal end on opposing ends of the longitudinal axis. Thereafter, the tubular tissue is split along a line that is parallel to the longitudinal axis to form a sheet, and the distal edge and the proximal edge are approximated towards each other to form an elongated graft element. The elongated graft element has a longitudinal axis that comprises the circumferential orientation of the tubular tissue, with the circumferential orientation being transverse to the longitudinal axis of the tubular tissue.

### BACKGROUND OF THE INVENTION

1. Field of the Invention

The present invention relates to prostheses and methods for implantation into a mammal, and in particular, to repair and/or reconstruct weakened or damaged connective tissue, such as ligament, tendon, and other defects. More particularly, the present invention relates to vascular tissue, dura mater, and pericardium used as a graft element for treatment of ligament, tendon and other deficiencies in a patient.

2. Description of the Prior Art

Men and women who are athletically active experience the majority of ligament tears, particularly tearing of the anterior cruciate ligament (ACL) of the knee. The ACL is commonly torn by forces applied to the knee during twisting, cutting, deceleration or tackling. A torn ACL will generally not heal. An ACL deficient knee is often unstable during pivoting activity. Repeated instability episodes of the knee may lead to further damage of the articular surface and cause tearing in the menisci. It is therefore desirable to stabilize the knee by reconstructing a torn ACL. Attempts in the past to directly repair the torn ACLs have been relatively ineffective.

One attempt has used prosthetic ligament replacements made of carbon fibers and Gore-Tex materials, but these prosthetic replacements do not last a long period of time. Repeated loading of a prosthetic ligament in a young active patient leads to failure of the ligament. The release of debris from a failed ligament results in chronic inflammation of the joint, and osteolysis of bone, in and around the area of ligament attachment.

Other attempts using heterogeneous tendon and ligament, small intestine submucosa tissue, synthetic material and tissue engineered ligaments usually do not provide optimal results due to (1) insufficient initial tension and strength, (2) poor long term graft flexibility, (3) excessive scar tissue formed around the graft, and (4) excessive adhesion on the graft surface. In this regard, good initial tension and strength are important characteristics that should be possessed by the material. For example, the material must be sufficiently strong to withstand continued bending and other flexing motions, and the material should have good initial tension to facilitate these bending and flexing motions.

The current standard practice is to reconstruct a torn ACL by substituting the torn ligament with a patient's own tissue. The middle third of the patellar tendon or the hamstring tendons are commonly used as substitution ligaments. Using a patient's own tissue is also associated with morbidity at the second surgery site. For example, stress fracture of the patellar, quadriceps muscle weakness and a long rehabilitation period may result from the use of a patient's own tissue. Furthermore, harvesting and preparation of autogeneous tissue prolong surgery time and cause additional trauma to the patient.

As an alternative, allograft patellar tendon, hamstring tendon or Achilles tendon from a donor can be used for reconstructing the ligament. However, donor materials carry a risk of infectious disease transmission.

### SUMMARY OF THE DISCLOSURE

It is an object of the present invention to provide a tissue graft for reconstruction or repair of previously torn ligaments and tendons or other body wall deficiencies.

It is another object of the present invention to provide a graft having different topographical properties on its surfaces, and different composition, so as to achieve desirable tissue adhesion and antiadhesion results.

In order to accomplish the objects of the present invention, the present invention provides a method of forming an elongated graft element that can be used to treat a torn tendon or ligament. According to the method, a tubular tissue is procured from a mammal, the tubular tissue having a longitudinal axis, and a distal edge and a proximal end on opposing ends of the longitudinal axis. Thereafter, the tubular tissue is split along a line from the distal edge to the proximal edge, and which is parallel to the longitudinal axis, to form a sheet. The distal edge and the proximal edge are then approximated towards each other to form an elongated graft element. The elongated graft element has a longitudinal axis that comprises the circumferential orientation of the tubular tissue, with the circumferential orientation being transverse to the longitudinal axis of the tubular tissue.

Thus, the present invention provides a tubular tissue and then processes the tubular tissue to form an elongated graft element that has a different orientation from the original orientation of the tubular tissue, so that the resulting elongated graft element can have sufficient strength and initial tension to be used as a graft for reconstruction or repair of previously torn ligaments and tendons or other body wall deficiencies.

### BRIEF DESCRIPTION OF THE DRAWINGS

FIG. 1 is a perspective view of a tubular tissue according to the present invention.

FIG. 2 illustrates the tubular tissue of FIG. 1 in a split form having a sheet-like configuration.

FIG. 3 illustrates a folded graft element formed from the sheet-like tissue of FIG. 2.

FIG. 4 illustrates a wrapped graft element formed from the sheet-like tissue of FIG. 2.

FIG. 5 illustrates a rolled graft element formed from the sheet-like tissue of FIG. 2.

FIG. 6 is a sectional view showing a tendon with a graft element according to the present invention adapted for bridging the torn ligament.

FIG. 7 is a sectional view showing the graft element of FIG. 6 being attached.

FIG. 8 is a simulated perspective view of a knee with a graft element of the present invention extending through the tibia and wrapped over the top of a femur.

FIG. 9 is a simulated perspective view of the repair of an articular capsule using a graft element of the present invention.

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

The present invention provides a tubular tissue and then processes the tubular tissue to form an elongated graft element that has a different orientation from the original orientation of the tubular tissue, so that the tissue can have sufficient strength and initial tension to be used as a graft for reconstruction or repair of previously torn ligaments and tendons or other body wall deficiencies.

The term “graft element” as used herein is intended to mean either a finished graft that is sized and shaped for implantation, or a component of a finished graft configured for implantation.

The term “tissue” as used herein is intended to mean any mammalian (human or animal) vascular (e.g., artery, vein), pericardium, dura mater, urethra, small intestine, colon, or similar tissue that has sufficient strength and flexibility to act as the primary component of the prosthesis. Tissue should have a cellular matrix of proteins (e.g., collagen). Tissue can be used as a partial thickness. Tissue can include tissue that is obtained from the host patient in which the prosthesis is to be implanted (known as autologous tissue), in which case the living cells inherited from the autologous tissue are generally maintained. Tissue can also include homologous tissue, such as from cadavers, umbilical cords, and placenta, in which case the cells are either dead or removed from the tissue. Such homologous tissue would be substantially devoid of an adventitial layer, and devoid of endothelial cells. In addition, tissue can include heterologous tissue, such as from porcine, bovine, canine, ovine, equine, etc, in which case the tissue is generally devoid of living cells. In one embodiment of the present invention, luminal or tubular tissues (e.g., venous tissue such as vena cava) are preferred. The tissue can be chemically treated or crosslinked (e.g., by glutaraldehyde, polyepoxy, PEG, etc.) or not chemically crosslinked (e.g., fresh, frozen, regenerated, tissue engineered, UV, heat, or cryopreserved). The tissue can also be chemically modified with proper charge and hydrophilicity. The tissue can be harvested according to known techniques, such as those described in Love, Autologous Tissue Heart Valves, R. G. Landes Co., Austin, Tex., 1993, Chapter 8. The tissue can also contain drugs, such as growth factor, antiadhesion drug, antibiotics, heparin, aspirin, etc.

The tissue in this invention can have different surface chemical composition and topographic characteristics. For example, the tissue can have two different surfaces, a smooth surface (e.g. the luminal surface or tunica intima of vascular tissue, or the serasol surface of pericardium), and a rough surface (e.g., the adventitial surface or tunica adventitia of vascular tissue, or the epipericardial surface of pericardium). The smooth surface has been shown to be highly useful in discouraging mesenchymal cell adhesion. Heparin is also known to minimize the cell adhesion. On the other hand, the rough surface of the tissue material promotes the attachment and proliferation of fibroblast cells and is important to good fibrous tissue adhesion and healing, and is therefore particularly suitable for entrapping and enhancing autologous cell growth once implanted as a graft element for treatment of ligament, tendon and other deficiencies. As explained in greater detail below, the rough surface can be configured to be an external surface of the graft element, and the smooth surface can also be configured to be an external surface of the graft element.

The term “mammal” as used herein can include porcine, bovine, equine, ovine, and human.

The term “drug” as used herein is intended to mean any compound which has a desired pharmacologic effect. The drug is preferably compatible with the tissue and can be tolerated in a patient. For example, the drug can be an anticoagulant, such as an RGD peptide-containing compound, heparin, antithrombin compounds, platelet receptor antagonists, anti-thrombin antibodies, anti-platelet receptor antibodies, aspirin, prostaglandin inhibitors, platelet inhibitors, or tick anti-platelet peptide. The drug can also be a promoter of vascular cell growth, such as a growth factor receptor antagonist, transcriptional activator or translational promoter. Alternatively, the drug can be an inhibitor of vascular cell growth, such as a growth factor inhibitor, growth factor receptor antagonist, transcriptional repressor or translational repressor, antisense DNA, antisense RNA, replication inhibitor, inhibitory antibodies, antibodies directed against growth factors, and bifunctional molecules.

The drug can also be a cholesterol-lowering agent, a vasodilating agent, and agents which interfere with endogenous vasoactive mechanisms. Other examples of drugs can include anti-inflammatory agents, anti-platelet or fibrinolytic agents, anti-neoplastic agents, anti-allergic agents, anti-rejection agents, anti-microbial or antibacterial or anti-viral agents, hormones, vasoactive substances, anti-invasive factors, anti-cancer drugs, antibodies and lymphokines, anti-angiogenic agents, radioactive agents and gene therapy drugs, among others. The drug may be loaded as in the drug's original commercial form, or together with polymer or protein carriers, to achieve delayed and consistent release.

Specific non-limiting examples of some drugs that fall under the above categories include paclitaxel, docetaxel and derivatives, epothilones, nitric oxide release agents, heparin, aspirin, coumadin, PPACK, hirudin, polypeptide from angiostatin and endostatin, methotrexate, 5-fluorouracil, estradiol, P-selectin Glycoprotein ligand-1 chimera, abciximab, exochelin, eleutherobin and sarcodictyin, fludarabine, sirolimus, tranilast, VEGF, transforming growth factor (TGF)-beta, Insulin-like growth factor (IGF), platelet derived growth factor (PDGF), fibroblast growth factor (FGF), RGD peptide, beta or gamma ray emitter (radioactive) agents.

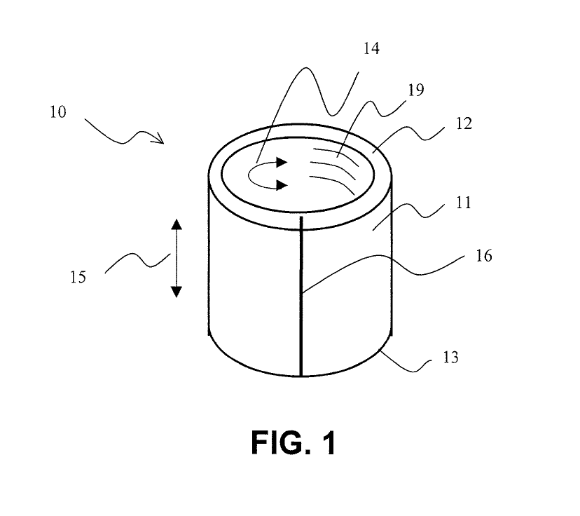

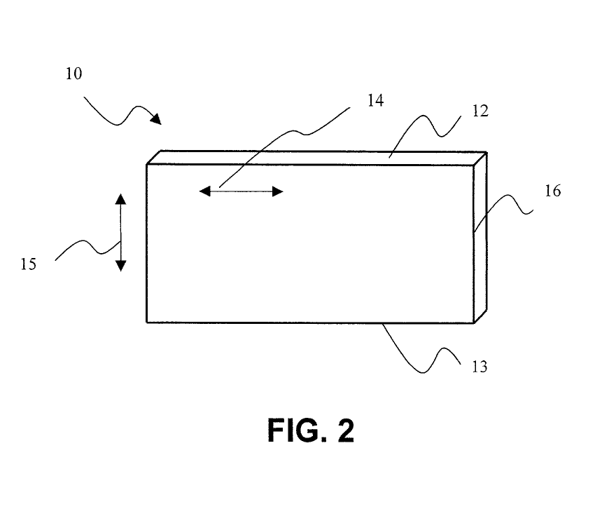

FIGS. 1 and 2 illustrate a first embodiment of the present invention, which provides a graft element suitable for ligament or tendon grafting. In FIGS. 1 and 2, a tubular tissue 10, such as a polyepoxy crosslinked bovine vena cava, has a longitudinal orientation 15 and a transverse circumferential orientation 14. The tubular tissue 10 has a distal edge 13 and a proximal edge 12 that are perpendicular to the longitudinal axis that is parallel to the longitudinal line 16. The tubular tissue 10 has a luminal surface 19 and an adventitial surface 11. Unfortunately, the strength of the tubular tissue 10 when stretched in the direction of the longitudinal orientation 15 is not sufficient for use as a graft element suitable for ligament or tendon grafting.

As a result, the tubular tissue 10 is split along a straight longitudinal line 16 (that is parallel to the longitudinal orientation 15 of the tubular tissue 10) from the distal edge 13 to the proximal edge 12, to form an essentially rectangular sheet as shown in FIG. 2. Configurations other than a rectangular shape can also be used depending on the desired applications, including but not limited to square, polygon, trapezoid, among others. The sheet shown in FIG. 2 is then folded, wrapped or rolled so that the distal edge 13 is approximated towards the proximal edge 12 to form an elongated graft element. The present inventor has found that the strength of the folded, wrapped or rolled sheet of tubular tissue 10 in the direction of the transverse circumferential orientation 14 is sufficiently strong, yet has sufficient initial tension, to be well-suited for use as a graft element for ligament or tendon grafting.

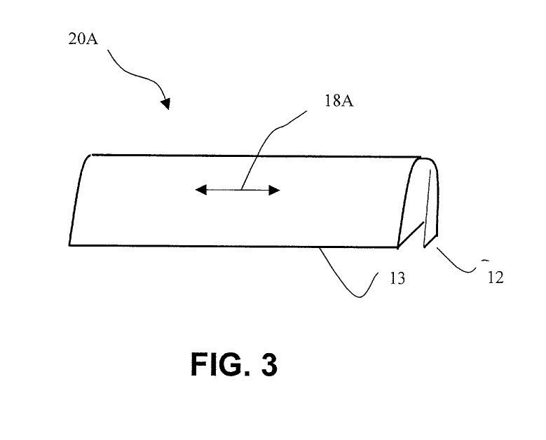

FIGS. 3-5 illustrate different ways of folding, wrapping or rolling so that the distal edge 13 is moved towards the proximal edge 12 to form an elongated graft element. Referring first to FIG. 3, the two edges 12 and 13 may be folded towards each other, and secured to each other, to form an elongated graft element 20A that has a longitudinal axis 18A that is essentially the transverse circumferential orientation 14 of the pre-split tubular tissue 10. The edges 12 and 13 can be secured to each other by stapling, suturing, adhering, welding, gluing and similar techniques, to form a tubular element. If the folded graft element shown in FIG. 3 is given another fold, the graft element will have four layers. Thus, a multiple layered graft element can be formed by repeatedly folding the edges 12 and 13.

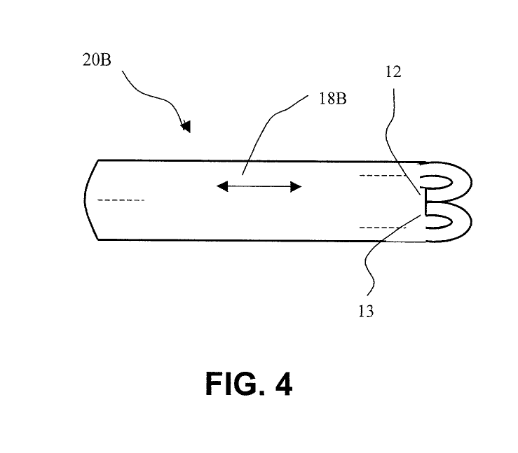

Referring now to FIG. 4, the two edges 12 and 13 may be wrapped towards each other so that the two edges 12 and 13 are side-by-side to each other when they are secured to each other. By wrapping the two edges 12 and 13 in the manner shown in FIG. 4, an elongated graft element 20B can be formed that it also has a longitudinal axis 18B that is essentially the transverse circumferential orientation 14 of the pre-split tubular tissue 10. The edges 12 and 13 can be secured to each other by stapling, suturing, adhering, welding, gluing and similar techniques, to form a tubular element. As with the embodiment of FIG. 3, a multiple layered graft element can be formed by repeatedly wrapping the edges 12 and 13.

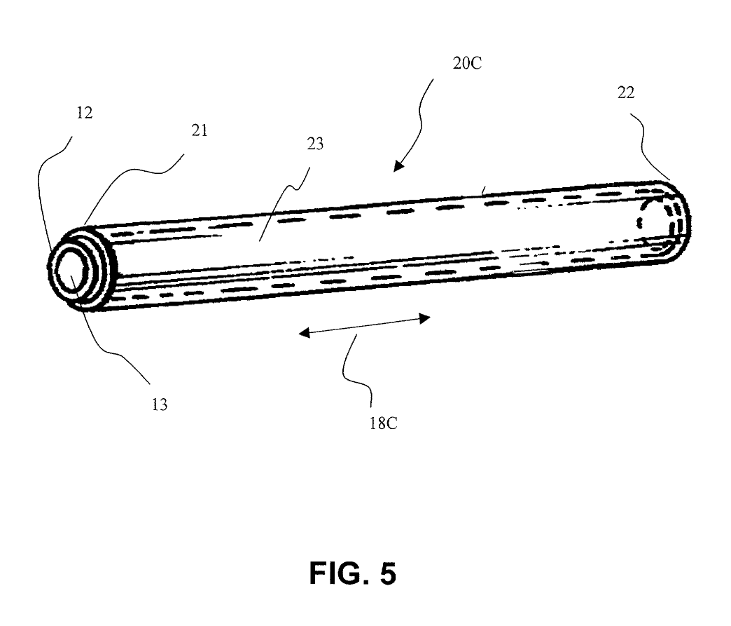

Referring now to FIG. 5, the two edges 12 and 13 may be rolled (e.g., around a temporary mandrel) to form a multiple layered, elongated graft element 20C that has a longitudinal axis 18C that is essentially the transverse circumferential orientation 14 of the pre-split tubular tissue 10. The edges 12 and 13 can be secured to each other by stapling, suturing, adhering, welding, gluing and similar techniques, to form a tubular element. The graft element 20C forms an elongated graft body 23 having a distal end 21 and a proximal end 22.

Thus, as shown in FIGS. 3-5, the orientation of the tissue 10 is changed so that the tissue 10 in its new orientation (i.e., 14) now experiences greater strength and better initial tension. Another way of viewing the present invention is that it changes the longitudinal axis of the tubular tissue 10, by making the transverse circumferential orientation 14 the new longitudinal axis.

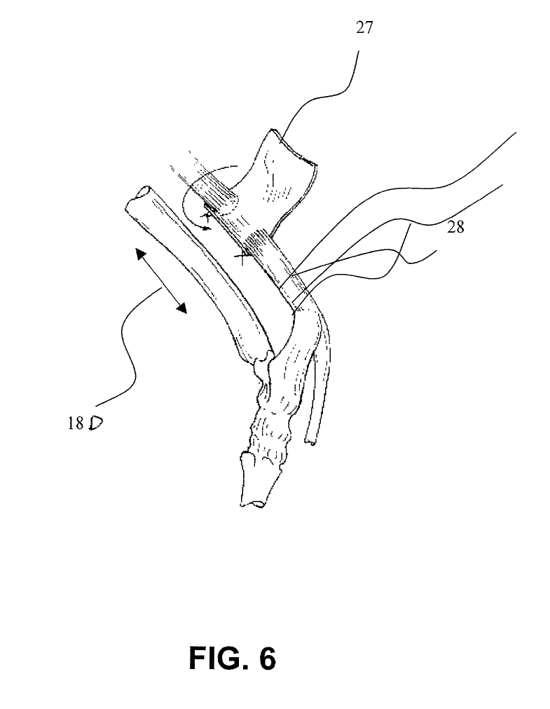

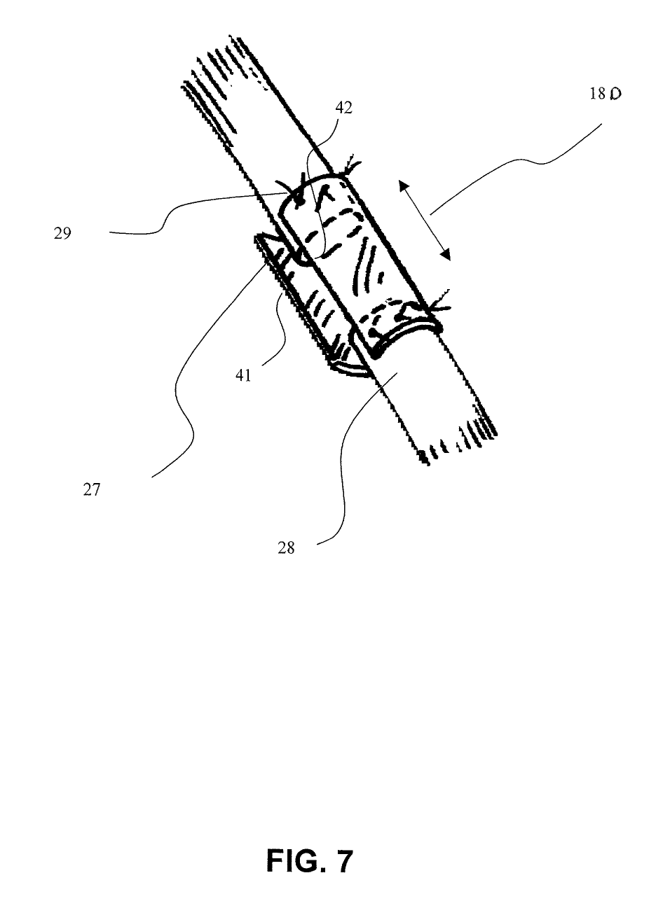

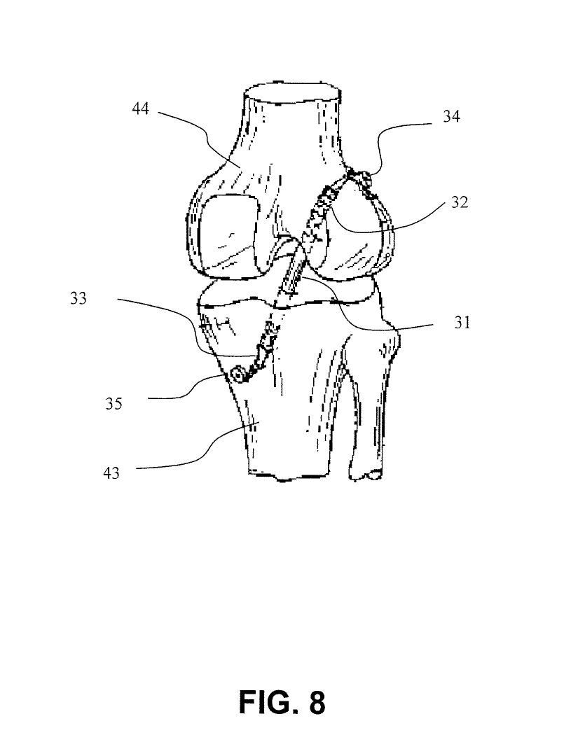

In one embodiment, the adventitia surface 11 is preferably kept to the outside of the graft 20A, 20B, 20C. This graft 20A, 20B, 20C can be used for tendon or ligament repair (FIGS. 6 and 7), or in ligament reconstruction (FIG. 8), as explained below. As an alternative, the adventitia surface 11 can be kept to the inside of the graft 20A, 20B, 20C and the smooth luminal surface is kept to the outside of the graft if the graft is being used for finger or hand tendon applications, where the smooth luminal surface 19 is effective in anti-adhesion.

In another embodiment, a polyepoxy crosslinked bovine vena cava (such as those described above) can be used to repair hernia. The luminal surface 19 of this tissue is kept towards the abdominal cavity to prevent the adhesion between the graft and the internal organs, while the adventitial surface 11 provides adhesion.

FIGS. 6 and 7 show how a graft element of the present invention (such as the graft element illustrated in FIG. 2 above) may be shaped and formed to connect a broken or severed achilles tendon. In one embodiment, the sheet-like elongate graft element 27 from FIG. 2 has a longitudinal axis 18D that corresponds to the transverse circumferential orientation 14 of the split tubular tissue 10. The graft element 27 is wrapped about the severed ends of the achilles tendon as shown in FIG. 7, and sutured (e.g., see sutures 29) to these severed ends of the tendon. In addition, one edge 41 of the tissue 10 can be sutured or otherwise secured to the opposing edge 42 of the tissue 10.

FIG. 8 is a simulated perspective view of a knee with a graft element extending through the tibia 43 and wrapped over the top of a femur 44. The graft element 31 can be an ACL graft element, and can be provided according to any of the embodiments illustrated above in FIGS. 3-5. The graft element 31 is implanted through tunnels 32 and 33 in the femur 44 and the tibia 43, respectively, and the graft element 31 is secured to the adjacent bones 34, 35. FIG. 8 illustrates that the graft element 31 is screwed to the adjacent bones 34, 35, but other known fixation methods can also be used.

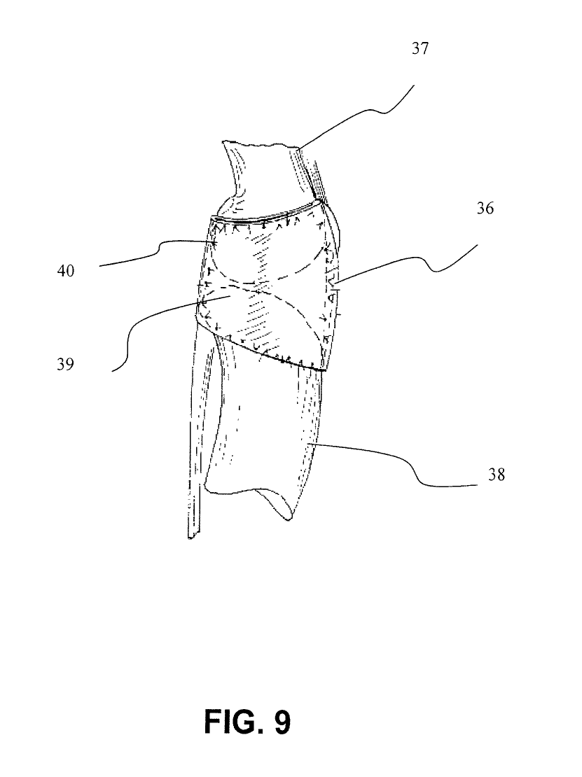

FIG. 9 is a simulated perspective view of the repair of an articular capsule using a graft element 36 that can be made according to FIG. 2 above. The sheet graft element 36 is sutured along sutures 40 to the adjacent bones 37, 38, with the rough surface 39 of the graft element 36 facing exteriorly from the joint capsule for enhancing tissue ingrowth. In other words, the smooth surface of the graft element 36 faces towards the inside.

Thus, the present invention provides a method for preparing an elongated graft element, where the method includes the steps of procuring a tubular tissue (as described above) from a mammal, splitting the tubular tissue along its longitudinal orientation to form a sheet, and then creating an elongated graft element having a new longitudinal axis that was previously the transverse circumferential orientation.

The present invention also provides a method for preparing a biomaterial having an elongated graft element. The biomaterial can be an implant such as a ligament, a tendon, a hernia supporter, a bladder sling, an organ compressor, an organ enforcer, or the like. The ultimate strength of the biomaterial is preferably higher than that of a natural ligament. The biomaterial can be loaded with drug. The biomaterial can be chemically treated by glutaraldehyde, formaldehyde, polyepoxy, PEG, or the like. Alternatively, the biomaterial is non-chemically treated by UV or heat.

While the description above refers to particular embodiments of the present invention, it will be understood that many modifications may be made without departing from the spirit thereof. The accompanying claims are intended to cover such modifications as would fall within the true scope and spirit of the present invention.

What is claimed is:

1. A method of forming an elongated graft element, comprising:
procuring a tubular tissue from a mammal, the tubular tissue having a longitudinal axis, and a distal edge and a proximal end on opposing ends of the longitudinal axis;
splitting the tubular tissue along a line from the distal edge to the proximal edge that is parallel to the longitudinal axis to form a sheet; and
approximating the distal edge and the proximal edge towards each other to form an elongated graft element.

2. The method of claim 1, further including:
attaching the elongated graft element to the severed ends of a tendon or ligament.

3. The method of claim 1, wherein the tubular tissue also has a circumferential orientation that is transverse to the longitudinal axis of the tubular tissue, and wherein the elongated graft element has a longitudinal axis that comprises the circumferential orientation.

4. The method of claim 1, wherein approximating the distal edge and the proximal edge towards each other comprises the step of wrapping the distal and proximal edges.

5. The method of claim 1, wherein approximating the distal edge and the proximal edge towards each other comprises the step of folding the distal and proximal edges.

6. The method of claim 1, wherein approximating the distal edge and the proximal edge towards each other comprises the step of rolling the sheet.

7. The method of claim 1, wherein the tubular tissue is a blood vessel.

8. The method of claim 1, wherein the tubular tissue is chemically treated.

9. The method of claim 7, wherein the tubular tissue is devoid of endothelial cells.

10. The method of claim 7, wherein the tubular tissue is devoid of an adventitial layer.

11. The method of claim 1, wherein the tubular tissue has a smooth surface and an opposing rough surface.

12. The method of claim 11, wherein the rough surface is configured to be an external surface of the elongated graft element.

13. The method of claim 11, wherein the smooth surface is configured to be an external surface of the elongated graft element.

14. The method of claim 1, wherein the distal and proximal edges are secured to each other.

15. The method of claim 14, wherein the distal and proximal edges are secured to each other using a technique selected from the group consisting of stapling, suturing, adhering, welding and gluing.

16. A method of forming an elongated graft element, comprising:
procuring a tubular tissue from a mammal, the tubular tissue having a longitudinal axis and a circumferential orientation that is transverse to the longitudinal axis;
splitting the tubular tissue along a line from the distal edge to the proximal edge that is parallel to the longitudinal axis to form a sheet; and
forming an elongated graft element that has a longitudinal axis that comprises the circumferential orientation.

17. The method of claim 16, wherein the longitudinal axis of the tubular tissue has a distal edge and a proximal end on opposing ends of the longitudinal axis of the tubular tissue, and wherein the step of forming an elongated graft element comprises approximating the distal edge and the proximal edge towards each other to form an elongated graft element.
